(19) United States
(12) Patent Application Publication
(10) Pub. No.: US 2006/0241351 A1
(43) Pub. Date: Oct. 26, 2006

(54) Integrated optical device for luminescence sensing

(75) Inventors: Chang-Dong Feng, Long Beach, CA (US); Barry W. Benton, Orange, CA (US)

Correspondence Address: WESTMAN CHAMPLIN & KELLY, P.A., SUITE 1400, 900 SECOND AVENUE SOUTH, MINNEAPOLIS, MN, 55402-3319, US

(73) Assignee: Rosemount Analytical Inc., Irvine, CA (US)

(21) Appl. No.: 11/398,789

(22) Filed: Apr. 6, 2006

Related U.S. Application Data
(60) Provisional application No. 60/669,650, filed on Apr. 8, 2005.

(51) Int. Cl. A61B 1/32

(52) U.S. Cl. 600/213

(57) ABSTRACT

An integrated luminescence sensor includes a light pipe in optical communication with a luminescence sensing layer. A source of excitation illumination is coupled to the light pipe and disposed to direct excitation illumination toward the sensing layer. A luminescent light detector is also coupled to the light pipe and is disposed to detect luminescent illumination luminescing from the sensing layer, which luminescence is related to interaction between the sensing layer and a substance of interest.

### CROSS-REFERENCE TO RELATED APPLICATION

The present application is based on and claims the benefit of U.S. provisional patent application Ser. No. 60/669,650, filed Apr. 8, 2005, the content of which is hereby incorporated by reference in its entirety.

### BACKGROUND OF THE INVENTION

Optical techniques for sensing substances of interest are known. One such technique is known as luminescent sensing. In luminescence sensing, a source of excitation illumination is provided and directed to a specialized sensing layer. The sensing layer has a sensing characteristic in that the sensing layer is quenched by the substance of interest. For example, the use of ruthenium (II) complexes for such sensors is known. The use of such ruthenium complexes for sensing oxygen is described in the art, for example see U.S. Pat. No. 4,752,115.

Much research has been directed to the various dyes and chemical complexes that can be used for the sensing layer of such luminescence sensors. However, an equally important consideration is that of the actual sensor technologies and configurations used to generate and sense the luminescence. Providing a sensor that could make better use of any luminescence sensing material, whether now known or later developed, would represent a significant advance in the art.

### SUMMARY

An integrated luminescence sensor includes a light pipe in optical communication with a luminescence sensing layer. A source of excitation illumination is coupled to the light pipe and disposed to direct excitation illumination toward the sensing layer. A luminescent light detector is also coupled to the light pipe and is disposed to detect luminescent illumination luminescing from the sensing layer, which luminescence is related to interaction between the sensing layer and a substance of interest.

### BRIEF DESCRIPTION OF THE DRAWINGS

FIG. 1 is a diagrammatic view of a prior art luminescence-based sensing system.

FIG. 2 is a diagrammatic view of a luminescence-based sensing system in accordance with an embodiment of the present invention.

### DETAILED DESCRIPTION

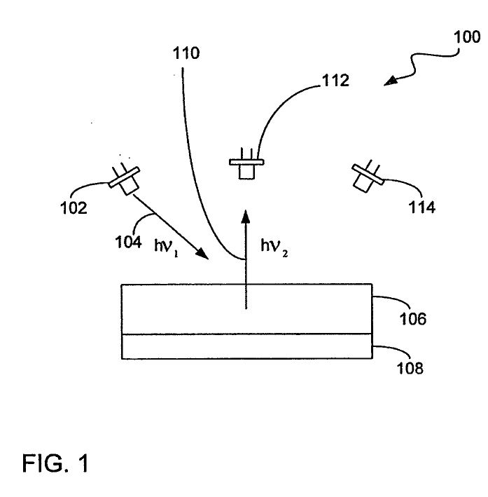

FIG. 1 is a diagrammatic view of an exemplary optical luminescence sensor 100. Sensor 100 includes excitation source 102 that is illustrated as a light emitting diode. Source 102 generates excitation illumination 104 that passes through transparent substrate 106 and interacts with sensing layer 108. When sensing system 100 is an oxygen sensor, sensing layer 108 may generally include a ruthenium complex dye, which dye is quenched by oxygen, which varies the degree to which the sensing layer luminesces. The luminescence illumination emanating from the sensing layer is sensed by luminescence light detector 112, which is used to measure the luminescence and ultimately provide an indication of oxygen concentration or partial pressure. Excitation light source 102 emits light Hυ₁ which excites the ruthenium complex luminescence dye molecules in sensing layer 108. The excited dye molecules then emit luminescence light Hυ₂, which is then measured by luminescent light detector 112. Sensing system 100 also generally includes excitation illumination detector 114 which is used to measure characteristics of the excitation illumination in order to compensate for changes therein. System 100 can be used for sensing oxygen when the dye molecule is, for example, a ruthenium based complex. In the presence of oxygen, the excited dye molecules will transfer energy to oxygen molecules instead of emitting the luminescent light. By measuring luminescent light, many oxygen sensing devices have been developed.

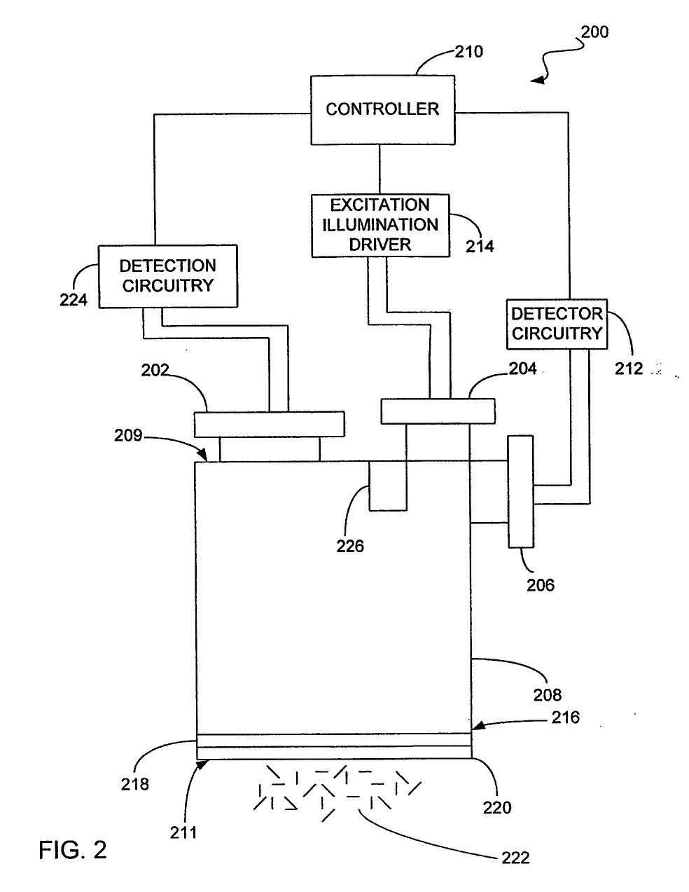

FIG. 2 is a diagrammatic view of luminescent quenching sensing system in accordance with an embodiment of the present invention. System 200 can be used to sense any substance for which a luminescent dye can be provided. System 200 includes luminescent light detector 202, excitation light source 204, and excitation light detector 206. Detector 202, source 204, and detector 206 are all mounted to light pipe 208. Sensing to system 200 includes controller 210 which may be any suitable processing circuit, such as a microprocessor. Controller 210 is coupled to detection circuitry 212 and to driver circuitry 214. Driver circuitry 214 is adapted to receive a signal from controller 210 and generate suitable energization signals to excitation light source 204. Some of the excitation light is detected by excitation light detector 206 via detector circuitry 212. The excitation illumination travels within light pipe 208 from first end 209 to sensing portion 216 disposed at second end 211. Sensing portion 216 includes transparent substrate 218 and sensing layer 220 comprising luminescent dye such as ruthenium complex luminescent dye. In accordance with known principles, interaction of substance 222 of interest with luminescence sensing layer 220 quenches sensing layer 220 and reduces its luminescence based on the degree of interaction, i.e. concentration, between substance 222 of interest and sensing layer 220. The luminescent illumination emanating from sensing layer 220 passes within light pipe 208 to luminescent light detector 202. Luminescent light detector 202 is coupled to detection circuitry 224 which is able to measure a property of luminescent light detector 202 related to the intensity of the luminescent illumination. Detection circuitry 224 provides a signal or data to controller 210 based upon the intensity of luminescent illumination.

Light pipe 208 can be any optically clear solid or fluid material that is able to suitably convey illumination therein. Light pipe 208 preferably has a circular cross section, but can have any suitable shape. Light pipe 208 preferably has a refractive index between about 1.0 to about 1.7. When solid material is used, all components are attached to the light pipe in such a way that there are no air or gaseous gaps between the components and the light pipe. This is so regardless of whether optical adhesive is used to attach the components. When fluid material is used within light pipe 208, all components are in contact with the light pipe filled in a vessel.

Sensing system 200 also includes blocking member 226 that is disposed to prevent excitation illumination from passing directly from excitation illumination source 204 to luminescence detector 202. Blocking member 226 may also be disposed to reflect a portion of excitation illumination from excitation illumination source 204 to excitation illumination detector 206. In the embodiment illustrated in FIG. 2, blocking member 226 is mounted within light pipe 208 proximate first end 209. However, blocking member 226 can be any suitable device that is able to prevent excitation illumination from passing directly from source 204 to detector 202. While the embodiment illustrated in FIG. 2 shows detector 202 and source 204 mounted next to each other proximate first end 209, they can be arranged in any suitable fashion including, without limitation, providing the excitation illumination source as a ring-shaped light source, with excitation source 204 disposed about luminescence detector 202. Further, excitation source 204 can be any generator of electromagnetic energy, which may be visible or not, and which may be structured or unstructured. In an embodiment where the excitation illumination source is disposed about the luminescence detector, blocking member 226 is preferably ring-shaped as well, thereby effectively blocking excitation illumination from passing directly from source 204 to detector 202.

The luminescent light from sensing layer 220 is a scattering light; it emits in all directions. To collect the luminescent light efficiently, preparation of the light pipe surface is advantageous. Light pipe 208 preferably has a polished internal surface. Additionally, since light pipe 208 has a refractive index that is higher than air, light pipe 208 can be used in air, since some of the luminescent light can be directed to light luminescent light detector 202 by total internal reflection according to Snell's law. Moreover, the polished internal surface can be additionally coated with a reflective material so that substantially all light is reflected by the surface of light pipe 208. Further still, the surface of light pipe 208 can be coated with a material of a certain color, or surface preparation. Through spectral selection, the colored surface will absorb light of a certain frequency and reflect light of another frequency. For example, if the surface of light pipe 208 is painted orange, the surface will absorb blue light but reflect red light. These various surface preparations can be done to any and all surfaces of light pipe 208, or the surface preparation can be done with respect to defined portions of the light pipe leaving the remaining surface(s) with different preparation(s).

The adaptation or preparation of all or portions of surfaces of an optical luminescence based sensor in order to facilitate excitation and/or detection can be done with respect to any suitable sensor structure. For example, spectral selection has been described with respect to light pipe embodiments of the present invention, however, any suitable structure, including optical luminescence-based sensor of the prior art, can be adapted for enhanced spectral selection in accordance with embodiments of the present invention. Examples of spectral selection include optical components or surface features that reflect wavelengths of the luminescence illumination but absorb or inhibit illumination of other frequencies. Further, any suitable optical components can be employed to focus, or otherwise concentrate luminescence illumination upon luminescence detector 202

Embodiments of the present invention generally provide various optical components coupled to a light pipe. When a solid is used as the light pipe, there are no gaseous gaps between such components and the light pipe. Since all components are attached to the light pipe, optical stability of the device is improved against mechanical and thermal shock. Moreover, since all components are optically coupled with the light pipe without any gaps, signal loss due to Fresnel reflection is reduced. Further still, by using various surface preparations of light pipe 208, the collection of the luminescent light becomes more selective and efficient.

Although the present invention has been described with reference to preferred embodiments, workers skilled in the art will recognize that changes may be made in form and detail without departing from the spirit and scope of the invention.

What is claimed is:

1. A luminescence-based chemical sensor comprising:
a light pipe that is configured to convey light between a first end and a second end;
a source of excitation illumination mounted to the light pipe proximate the first end and disposed to direct excitation illumination towards the second end;
a sensing layer disposed proximate the second end, the sensing layer including a luminescent dye material that generates luminescent illumination within the light pipe in response to the excitation illumination, wherein the quantity of luminescent illumination is related to quenching of the luminescent dye material by a substance of interest; and
a luminescence illumination detector mounted to the light pipe proximate the first end and configured to generate an indication of luminescent illumination intensity.

2. The sensor of claim 1, wherein the light pipe is a transparent solid.

3. The sensor of claim 2, wherein the solid has in index of refraction between about 1.0 and about 1.7.

4. The sensor of claim 1, wherein the light pipe is a fluid.

5. The sensor of claim 4, wherein the fluid has an index of refraction between about 1.0 and about 1.7.

6. The sensor of claim 1, wherein the sensing layer includes a Ruthenium (II) complex luminescent dye.

7. The sensor of claim 1, and further comprising an excitation illumination detector mounted to the light pipe and disposed to measure a characteristic of the excitation illumination.

8. The sensor of claim 7, wherein the excitation illumination detector is mounted to the light pipe proximate the first end.

9. The sensor of claim 7, and further comprising a blocking member disposed to block excitation illumination from passing directly from the excitation illumination source to the excitation illumination detector without encountering an internal surface of the light pipe.

10. The sensor of claim 9, wherein the blocking member is mounted within the light pipe.

11. The sensor of claim 10, wherein the blocking member is mounted proximate the first end.

12. The sensor of claim 1, wherein the light pipe has a surface that is configured to improve light transmission within the light pipe.

13. The sensor of claim 12, wherein the surface is polished.

14. The sensor of claim 12, wherein the surface is colored to absorb light with certain frequencies and reflect light with other frequencies.

15. The sensor of claim 12, wherein the surface that is configured to improve light transmission includes substantially all of the surface of the light pipe.

16. The sensor of claim 12, wherein the surface that is configured to improve light transmission includes only a portion of the total internal surface of the light pipe.

17. A chemical sensing system comprising:
a luminescence-based chemical sensor including:
a light pipe that is configured to convey light between a first end and a second end;
a source of excitation illumination mounted to the light pipe proximate the first end and disposed to direct excitation illumination towards the second end;
a sensing layer disposed proximate the second end, the sensing layer including a luminescent dye material that generates luminescent illumination within the light pipe in response to the excitation illumination, wherein the quantity of luminescent illumination is related to quenching of the luminescent dye material by a substance of interest;
a luminescence illumination detector mounted to the light pipe proximate the first end and configured to generate an indication of luminescent illumination intensity; and
luminescence detector circuitry coupled to the luminescence illumination detector; driver circuitry coupled to the source of excitation illumination; and
a controller coupled to the luminescence detector circuitry and to the driver circuitry, the controller adapted to cause the driver circuitry to energize the source of excitation illumination and to receive an indication of luminescence intensity from the luminescence detector circuitry.

18. The sensing system of claim 17, and further comprising:
an excitation illumination detector mounted to the light pipe and disposed to measure a characteristic of the excitation illumination; and
excitation detector circuitry coupled to the excitation illumination detector and to the controller.

19. A luminescence-based chemical sensor comprising:
a source of excitation illumination disposed to direct excitation illumination towards a sensing layer;
a sensing layer including a luminescent dye material that generates luminescent illumination in response to the excitation illumination, wherein the quantity of luminescent illumination is related to quenching of the luminescent dye material by a substance of interest;
a luminescence illumination detector configured to generate an indication of luminescent illumination intensity; and
a surface within the sensor, wherein the surface is configured to enhance detection of the luminescence illumination.

20. The sensor of claim 19, wherein the surface has a color selected to reflect luminescence illumination and to absorb the excitation illumination.
